(19) United States
(12) Patent Application Publication
(10) Pub. No.: US 2016/0263346 A1
(43) Pub. Date: Sep. 15, 2016

(54) Device for well-being and body care

(71) Applicant: SPIRITUAL SUN DI CANOVESE GIULIANO, TAORMINA (ME) (IT)

(72) Inventor: Giuliano Canovese, Taormina (ME) (US)

(73) Assignee: SPIRITUAL SUN DI CANOVESE GIULIANO, Taormina (ME) (IT)

(21) Appl. No.: 15/029,842

(22) PCT Filed: Sep. 18, 2014
(86) PCT No.: PCT/IT2014/000250

§ 371 Date: Apr. 15, 2016

(30) Foreign Application Priority Data
Oct. 18, 2013 (IT) CT2013A000024

(51) Int. Cl. A61M 21/02; A45D 19/04

(52) U.S. Cl. CPC A61M 21/02; A45D 19/04; A45D 2019/005

(57) ABSTRACT

A device for well-being and body care includes a unit adapted to drip a fluid onto the body of a person to be treated and a unit collecting the fluid dripping from the body of the person, wherein such unit adapted to drip the fluid onto the body of the person to be treated, includes a tank connected, through a first duct, to a pump, the pump being then connected, through a second duct, to a percolator adapted to delicately flow the fluid onto the body of the person to be treated.

The present invention refers to a device for well-being and body care, adapted to let a heated fluid flow onto the forehead and the body of a person to be treated, in order to loosen his nervous tensions and create a relaxed state of his physique and of his mind.

With the current state of the art, in order to relax and create a well-being state of the body different techniques are used, based on gymnastics therapies, such as yoga, steam baths, massages with essential oils, etc. Well-being, beauty, psyco-physical balance, relax, mind and body harmony, are nowadays words which characterize and point out a new style of life.

All above listed therapies and different techniques, reducing stiffness, slowness and heaviness, dilate the body channels, creating an easier way for removing toxins.

Said techniques allow the body to acquire more flexibility and agility, for a very pleasant well-being and relaxing state; however, the mind remains active and relaxes with difficulty, particularly for those having an intense mind activity and frequently suffer from insomnia, stress and/or nervous wreckage. A mind bombarded by sense stimuli is subjected to a lot of stress and gets quickly fatigued, since worries consume more vital energy than physical tensions. A tired mind is then also one of the main causes of physical fatigue.

The present invention at least partly solves the problems of relaxing the mind, by providing a device for well-being and body care, according to claim 1, characterized in that it comprises means adapted to perform a continuous dripping of a fluid heated at a suitable temperature on the forehead of a person, and means adapted to heat said fluid at said temperature.

According to a preferred embodiment, the device according to the invention comprises means for collecting the fluid and is then advantageously contained into a small piece of furniture, assembled on wheels, which can be easily moved next to a suitable bed, so that the person subjected to treatment and resting supine onto said bed, can comfortably rest its neck back onto said means for collecting the fluid.

By providing said bed with suitable collecting means, it will be possible to use the same technique, by percolating heated essential oils, on the central and final part of the body.

The invented apparatus, due to heating of said fluid (for example cosmetic essential oils, sunflower oil, almond oil, olive oil, etc.), through a continuous dripping onto the forehead, variable between 20 and 50 minutes, regulates the emotional activity, taking to a very deep relaxing state and to a “mind emptiness” form, wherein, though remaining awake, one manages to “turn off” the mind and to relax the tensions in the whole body.

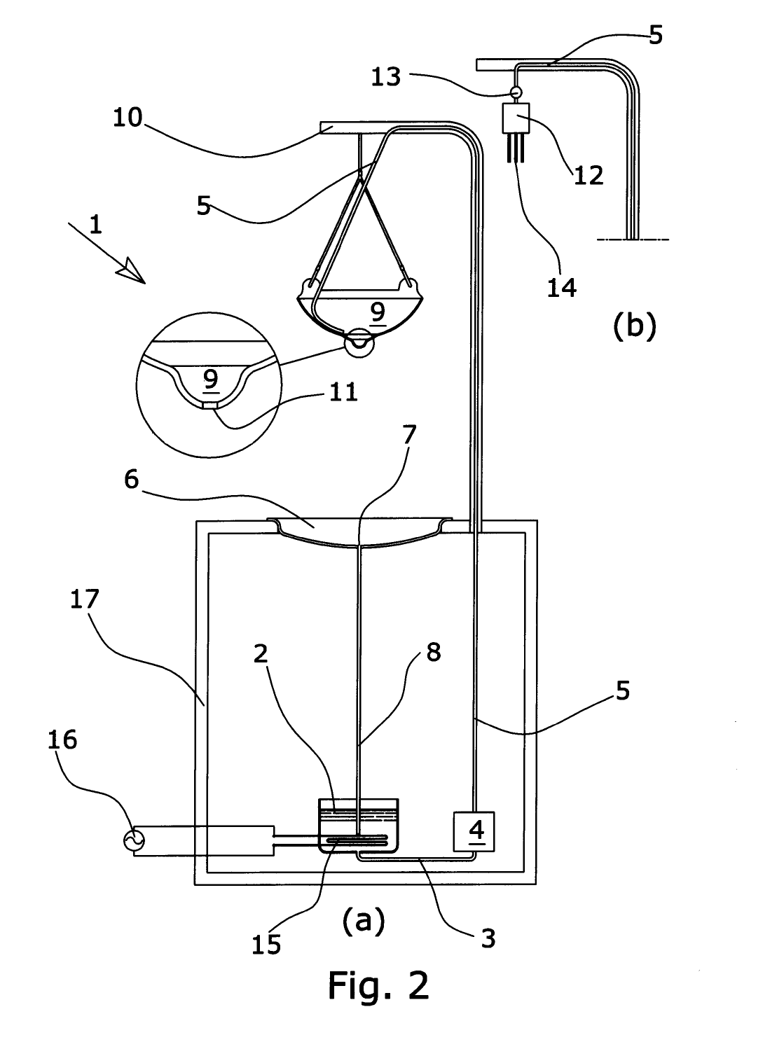

The oil dripping can be performed through a basin equipped with a hole, in which said oil is inserted, or through a small pump which supplies 3 or 4 small tubes. This latter solution will allow using the oil horizontally, vertically and rotatingly, as can be seen in FIG. 2. The solution with a single small tube is not shown.

At the end of the treatment, the apparatus can be used also for washing the hair, using said means for collecting fluid in order to collect water and shampoo used for washing, water being supplied by said dripping means or by a suitable shower head.

The invention will now be described, as a non-limiting example, according to a preferred embodiment thereof and with reference to the enclosed Figures, in which:

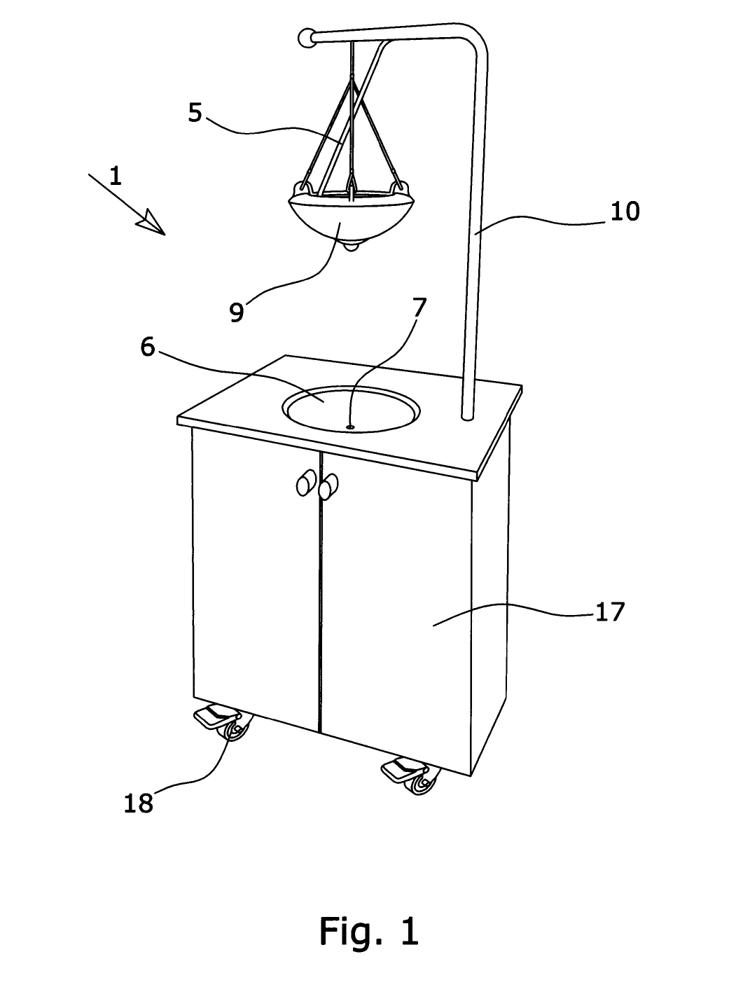

FIG. 1 shows the subject matter of the invention;

FIGS. 2(a, b) show the functional diagram of the invention;

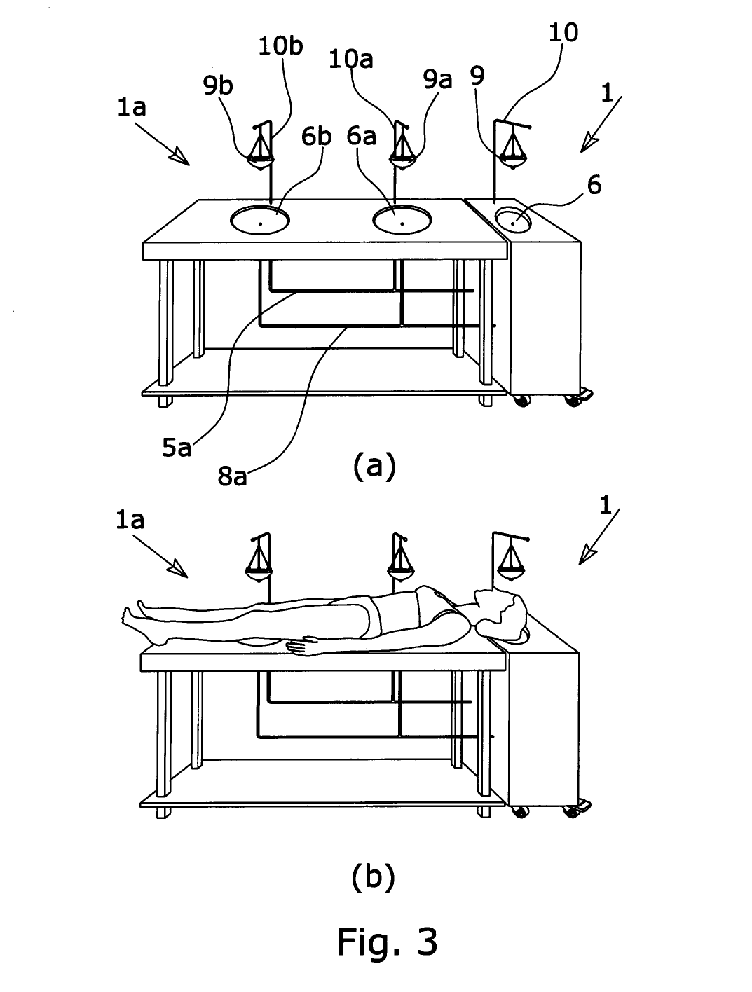

FIGS. 3(a, b) show a possible use of the invention together with a bed equipped with suitable collecting means.

With reference to FIGS. 1 and 2, 1 designates a device for well-being and body care according to the invention. Said device (1) essentially comprises means adapted to drip fluid onto the body of a person to be treated and means for collecting said fluid dripping from the body of said person. Said means, adapted to drip said fluid onto the head of the person to be treated, comprise a tank (2) connected through a first duct (3) to a pump (4), said pump (4) being then connected, through a second duct (5), to percolating means adapted to delicately flow said fluid onto the head of the person to be treated.

The fluid dripping from the person's head will then be collected by a basin (6), equipped with a discharge hole (7), from which fluid, passing through a third duct (8), is recycled into the tank (2).

Said percolating means, adapted to delicately flow said fluid onto the head of the person to be treated, comprise a basin (9), hanging from a support (10), in order to be at a short distance from the person's head, in which fluid coming from said second duct (5) is collected, said basin (9) being equipped with a hole (11), from which fluid goes out. According to a preferred embodiment, the diameter of the hole (11) is 3÷4 mm.

According to a preferred embodiment, shown in FIG. 2b, said percolating means can advantageously comprise a small motor (12), assembled onto said second duct (5) through a ball joint (13). In this way, it is possible to make fluid go out from at least one small tube (14), but also a plurality of small tubes (14), for example 3 or 4, and spray the fluid itself, in addition to vertically, also horizontally, by suitably orienting the joint (13), and, using the small motor (12), a rotary movement can also be given to the fluid.

According to a preferred embodiment, the position of said percolating means can be regulated in height, said regulation being adapted to occur by acting on small cord through which the basin (9) is hanging, or by regulating the height position of the support (10).

In order to improve the treatment efficiency, fluid can advantageously be heated at a suitable temperature, by using at least one resistance (15) (and preferably two, three or more resistances) connected to a source of electric energy (16). The temperature control could be made through a thermostat (not shown) as known in the art.

An electric cabinet (not shown) completes the device (1) according to the invention, for checking all functions, also in this case as known in the art.

The device (1) according to the invention is advantageously inserted into a small piece of furniture (17) assembled on four wheels (18), at least two of which equipped with brakes.

In this way, the device (1) can be easily transported on its place of use and it is possible, by operating on the brakes of the wheels (18), to secure it in its chosen position.

FIGS. 3(a, b) show a possible application of the device (1) according to the invention together with a bed (1a) equipped with a pair of small tanks (6a, 6b) and percolating basins (9a, 9b) hanging from their respective supports (10a, 10b). The percolating basins (9a, 9b) are supplied through a duct (5a) connected to the supply duct (5) of the percolating basin (9), while the discharge holes of the small tanks (6a, 6b) are connected, through a duct (8a), to the collecting tank (2).

Obviously, also in this case percolating means of the above described type can be used, which comprise the small motor (12), assembled onto a joint (13), which handles the small tubes (14).

With the described configuration of the device (1), it is possible to perform the treatment also on the median and lower parts of the body, as shown in FIG. 3b.

The described device is then advantageously completed by a system for washing the hair. This can be obtained by adding a shower head and using the small tank (6) or collecting soapy water, obviously by providing a deviation in the discharge duct (8), in order to avoid mixing soapy water with treatment fluid.

1. A device for well-being and body care comprising:
a unit adapted to drip a fluid onto a body of a person to be treated; and
a unit collecting said fluid dripping from the body of said person.

2. The device for well-being and body care according to claim 1, wherein said unit adapted to drip said fluid onto the body of the person to be treated, comprises a tank (2) connected, through a first duct (3), to a pump (4), said pump (4) being then connected, through a second duct (5), to a percolator (9, 14) adapted to delicately flow said fluid onto the body of the person to be treated.

3. The device for well-being and body care according to claim 2, wherein said percolator adapted to delicately flow said fluid onto the body of the person to be treated, comprises one or more basins (9, 9a, 9b) hanging from one or more supports (10, 10a, 10b), in order to be at in proximity of the body of the person, fluid coming from said second duct (5, 5a) being collected, said basins (9, 9a, 9b) being equipped with a hole (11), from which fluid goes out and drips onto the body of the person.

4. The device for well-being and body care according to claim 3, wherein said hole (11), made on said one or more basins (9, 9a, 9b), has a diameter included between 3 and 4 millimeters.

5. The device for well-being and body care according to claim 2, wherein said percolator adapted to delicately flow said fluid onto the body of the person to be treated, comprises at least one or a plurality of small tubes (14), assembled onto said second duct (5, 5a), from which said fluid goes out and delicately flows onto the body of the person to be treated.

6. The device for well-being and body care according to claim 5, wherein said small tubes (14) are assembled through a ball joint (13) in order to be oriented along directions which are different from vertical, in order to laterally treat a head and the body of the person.

7. The device for well-being and body care according to claim 5, wherein said percolator adapted to delicately flow said fluid onto the body of the person to be treated, further comprises a small motor (12), assembled onto said second supply duct (5, 5a) such that a rotary movement is given to the fluid, said fluid going out of said small tubes (14).

8. The device for well-being and body care according to claim 1, further comprising a shower head, placed next to a second basin (6), to be used for washing hair of the person.

9. The device for well-being and body care according to claim 8, further comprising a deviation in a discharge duct (8), in order to prevent mixing of soapy water with treatment fluid when washing hair with water and shampoo.

10. The device for well-being and body care according to claim 1, further comprising a piece of furniture (17) housing and/or supporting said units.

11. The device for well-being and body care according to claim 10, wherein said piece of furniture is assembled on wheels (18), in order to be easily transported to a place of use.

12. The device for well-being and body care according to claim 2, further comprising a bed (1a), equipped with:
the percolator (9a, 9b), hanging from a support (10a, 10b) and connected through a duct (5a) to said second duct (5);
a collecting basin (6a, 6b), connected through a duct (8a) to said tank (2);
said bed (1a) being adapted to support the person subjected to treatment with a head resting onto said collecting basin (6) and to perform a treatment also on median and lower parts of the body.

13. The device for well-being and body care according to claim 1, wherein said fluid comprises a cosmetic essential oil, sunflower oil, almond oil, or olive oil.

14. The device for well-being and body care according to claim 1, further comprising a system heating (15) and checking temperature of said fluid.

15. The device for well-being and body care according to claim 14, wherein said fluid is heated at a suitable temperature, through at least one resistance (15) connected to a source of electric energy (16).
